(19) United States
(12) Patent Application Publication
(10) Pub. No.: US 2014/0039501 A1
(43) Pub. Date: Feb. 6, 2014

(54) METHOD AND APPARATUS FOR PROVIDING A SOFT-TISSUE TRANSPLANT TO A RECEIVING BONE

(71) Applicant: THE CLEVELAND CLINIC FOUNDATION, Cleveland, OH (US)

(72) Inventors: Mark S. Schickendantz, Novelty, OH (US); Lutul D. Farrow, Tuscon, OH (US)

(73) Assignee: THE CLEVELAND CLINIC FOUNDATION, Cleveland, OH (US)

(21) Appl. No.: 13/960,247

(22) Filed: Aug. 6, 2013

Related U.S. Application Data
(60) Provisional application No. 61/679,817, filed on Aug. 6, 2012.

(51) Int. Cl. A61F 2/08; A61B 17/17

(52) U.S. Cl. CPC A61F 2/0811; A61B 17/1714
USPC 606/79; 623/13.14

(57) ABSTRACT

A method of providing a soft-tissue transplant to a receiving bone includes providing a transplant graft, comprising an elongated soft tissue, having first and second soft tissue ends longitudinally separated by a soft tissue body, and a bone block directly connected with the first soft tissue end. The bone block and soft tissue have been integrally formed as a unitary whole. An anchor cavity is machined in the receiving bone. The anchor cavity is shaped to substantially accept the bone block in a mating relationship. A majority of a volume of the bone block is placed in the anchor cavity. The bone block is fastened within the anchor cavity.

### RELATED APPLICATION

This application claims priority from U.S. Provisional Application No. 61/679,817, filed 6 Aug. 2012, the subject matter of which is incorporated herein by reference in its entirety.

### TECHNICAL FIELD

The present invention relates to a method and apparatus for providing a soft-tissue transplant to a receiving bone and, more particularly, to a method and apparatus for providing a medial ulnar collateral ligament transplant graft to an ulna.

### BACKGROUND OF THE INVENTION

Injuries to the medial ulnar collateral ligament (MUCL) are not uncommon. Classically, these injuries are seen in the overhead-throwing athlete. Many injuries to the MUCL may be treated nonoperatively with good results. A considerable subset of athletes may require operative reconstruction of the anterior band of the MUCL, though, if they are unable to return to their pre-injury level of function following conservative management.

The MUCL is the major soft tissue restraint to valgus stress of the elbow from 30 to 120 degrees flexion. This ligament serves an integral function in overhead-throwing athletes who experience repeated valgus loads across the elbow. The anterior band is the key anatomic component of the MUCL that resists valgus stress during the throwing motion.

Current surgical reconstructive techniques are designed to address the anterior band of the MUCL. The goals of surgery are to restore the normal biomechanics and kinematics of the elbow joint. While some surgeons have presented good to excellent long term results in over 90 percent of athletes with surgical reconstruction, other authors have been unable to reproduce these results and have lower success rates. A thorough understanding of the anatomy of the medial ulnar collateral ligament is critical for successful reconstruction of the MUCL. The soft tissue anatomy of the medial ulnar collateral ligament ulnar attachment has previously been described. However, the length of the ulnar attachment of the ligament was not described. No study describing the soft-tissue, osseous, and radiographic anatomy of the MUCL ulnar footprint is known to the inventors at this time, who hypothesize that the MUCL has a long soft tissue attachment on the proximal aspect of the ulna and that there is a consistent osseous ridge in this location that corresponds to the attachment of the anterior band of the MUCL.

### SUMMARY OF THE INVENTION

In an embodiment of the present invention, a method of providing a soft-tissue transplant to a receiving bone is described. A transplant graft is provided, comprising an elongated soft tissue, having first and second soft tissue ends longitudinally separated by a soft tissue body, and a bone block directly connected with the first soft tissue end. The bone block and soft tissue have been integrally formed as a unitary whole. An anchor cavity is machined in the receiving bone. The anchor cavity is shaped to substantially accept the bone block in a mating relationship. A majority of a volume of the bone block is placed in the anchor cavity. The bone block is fastened within the anchor cavity.

In an embodiment of the present invention, a system for providing a soft-tissue transplant to a receiving bone is provided. A transplant graft comprises an elongated soft tissue, having first and second soft tissue ends longitudinally separated by a soft tissue body, and a bone block directly connected with the first soft tissue end. The bone block and soft tissue have been integrally formed as a unitary whole. An anchor cavity is provided in the receiving bone. The anchor cavity is shaped to substantially accept the bone block in a mating relationship. A majority of a volume of the bone block is placed in the anchor cavity and the bone block is fastened to the receiving bone to place the soft tissue in a predetermined connected relationship with the receiving bone.

In an embodiment of the present invention, a method of providing a medial ulnar collateral ligament transplant graft to an ulna is provided. A transplant graft comprising an elongated ligament is provided, having first and second ligament ends longitudinally separated by a ligament body, and a bone block directly connected with the first ligament end. The bone block and ligament have been integrally formed as a unitary whole. An anchor cavity is machined in the ulna. The anchor cavity is shaped to substantially accept the bone block in a mating relationship. A majority of a volume of the bone block is placed in the anchor cavity. The bone block is fastened within the anchor cavity.

### BRIEF DESCRIPTION OF THE DRAWINGS

For a better understanding of the invention, reference may be made to the accompanying drawings, in which:

FIG. 1 is a side view of an example use environment for the present invention;

FIG. 2 is a side view of the use environment of FIG. 1 in a second configuration;

FIG. 3A is a perspective side view of a transplant graft according to an embodiment of the present invention;

FIG. 3B is a top view of the transplant graft of FIG. 3A;

FIG. 4A is a perspective side view of a cavity template according to an embodiment of the present invention;

FIG. 4B is a top view of the cavity template of FIG. 4A;

FIG. 5A is a schematic top view of the cavity template of FIG. 4A in the use environment of FIG. 1;

FIG. 5B is a schematic side view of the cavity template and use environment as shown in FIG. 5A;

FIG. 6 is a schematic perspective top view of the use environment of FIG. 1 in a third configuration;

FIG. 7 is a schematic side view of the embodiment of FIGS. 3A-4B in the use environment of FIG. 1;

FIGS. 8-19 schematically depict an example sequence of operation in the use environment of FIG. 1; and

FIGS. 20A-20B are side views of alternate embodiments of a component of FIG. 10.

### DESCRIPTION OF EMBODIMENTS

The patient tissue is shown and described herein at least as an elbow joint and the soft-tissue transplant is shown and described herein at least as a medial ulnar collateral ligament (MUCL), but the patient tissue and corresponding soft-tissue transplant could be any desired types such as, but not limited to, those used in hip joints, shoulder joints, knee joints, ankle joints, phalangeal joints, metatarsal joints, spinal structures, long bones (e.g., fracture sites), or any other suitable patient tissue use environment for the present invention. The below description presumes that the system, apparatus, and method described is being used in conjunction with a surgical procedure (namely, an at-least-partial MUCL replacement surgery), but the system(s), apparatus, and method(s) described may be used in any desired manner and for any desired purpose without harm to the present invention.

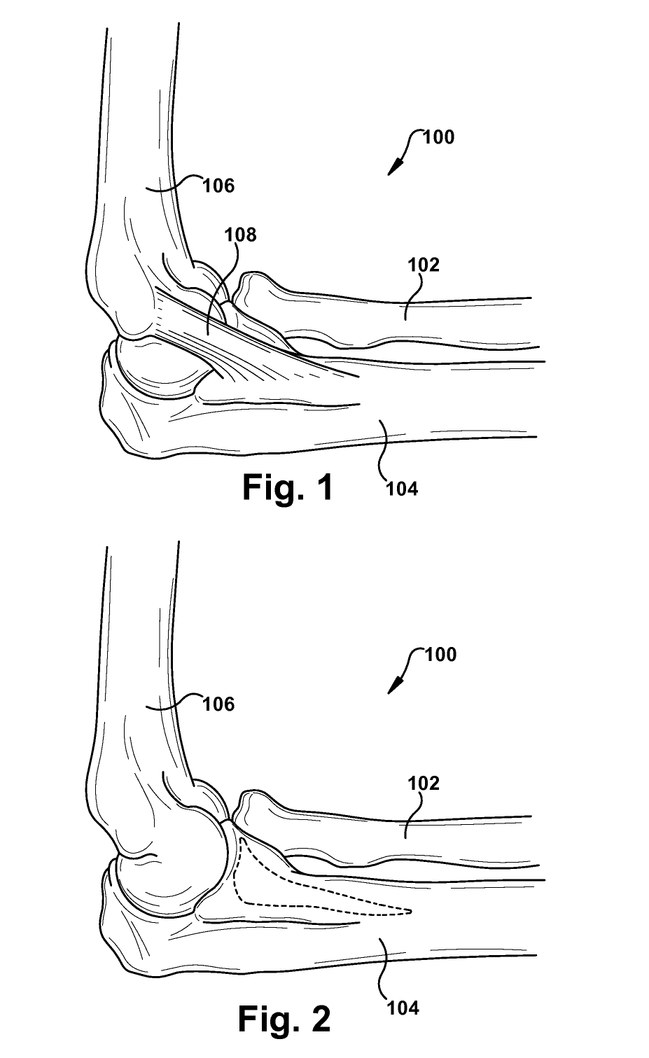

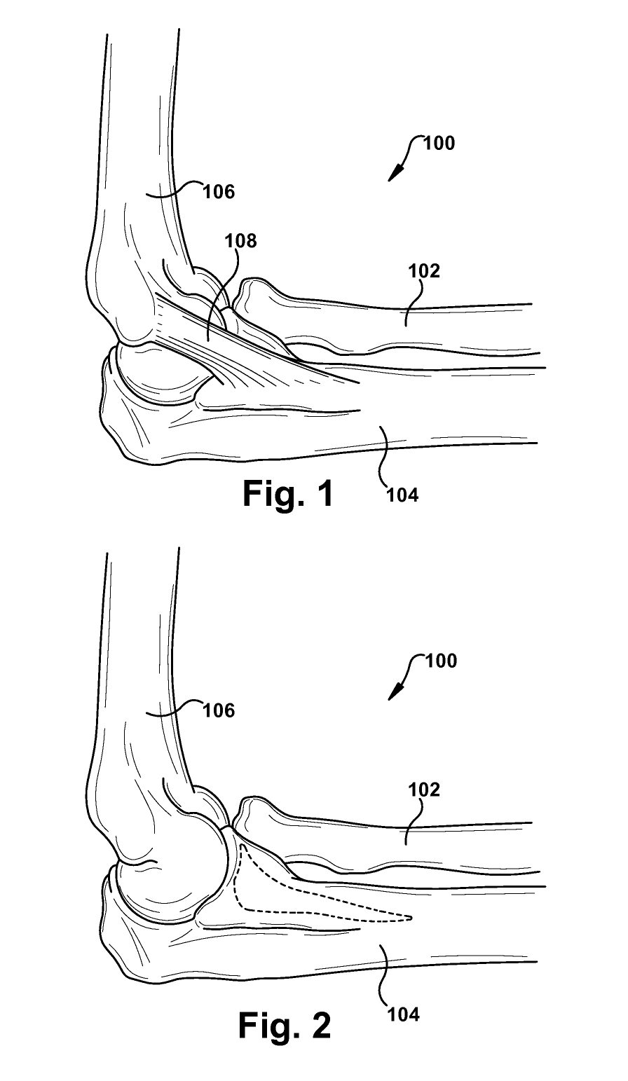

In accordance with the present invention, FIG. 1 depicts native tissue structures comprising an elbow joint 100 of a patient. A radius 102 and ulna 104 are attached to a humerus 106 by a MUCL 108. In FIG. 2, the native MUCL 108 has been removed preparatory to replacement by a soft-tissue transplant.

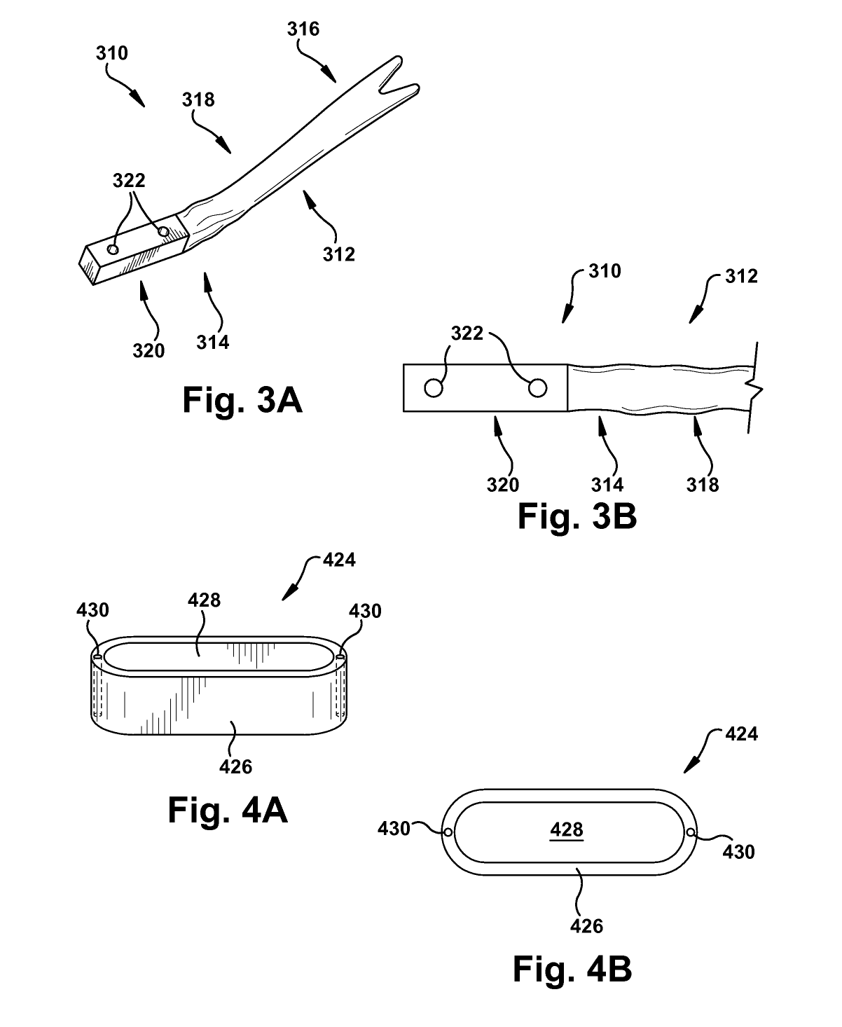

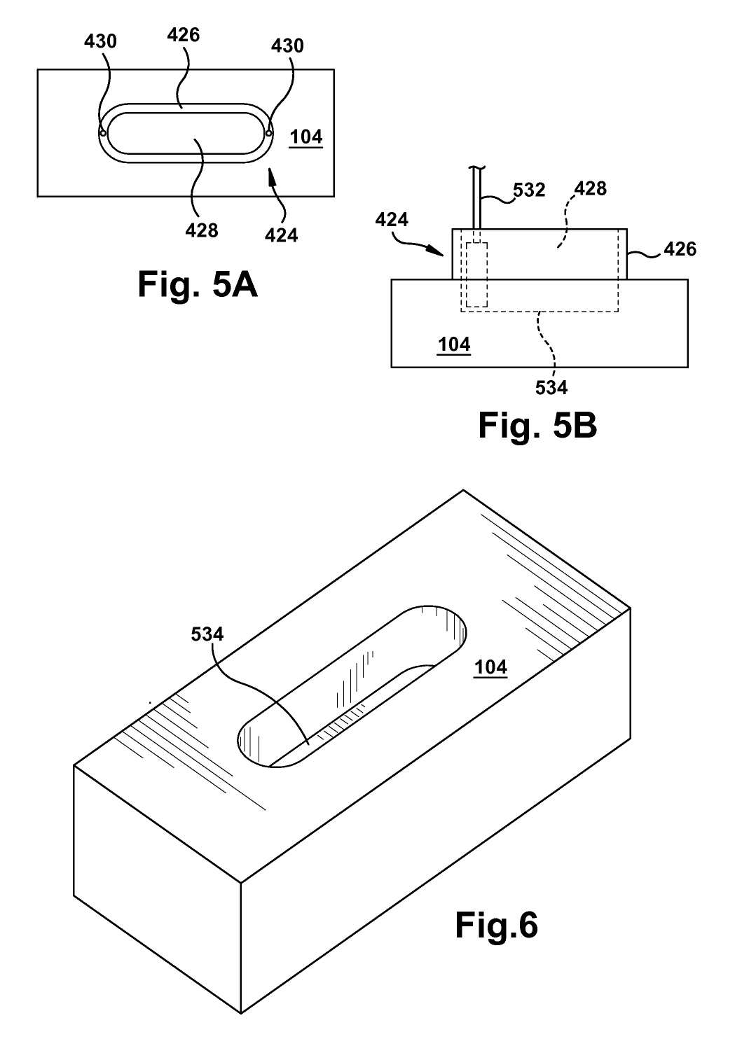

The transplant graft 310 is shown in FIGS. 3A-3B. An elongated soft tissue 312, from any suitable source, has first and second soft tissue ends 314 and 316, respectively, longitudinally separated by a soft tissue body 318. A bone block 320 is directly connected with the first soft tissue end 314. The bone block 320 and soft tissue 312 may have been integrally formed as a unitary whole. That is, particularly when the transplant graft 310 is taken as a single piece from a (living or dead) human or animal as an autologous graft, allogeneic graft, or any other type of heterograft or homograft, the bone block 320 could be cut from the donor during harvesting of soft tissue 312 (e.g., a ligament or tendon) that is inherently attached to the bone block in the native donor tissue as a feature of the native tissue anatomy. In other words, the transplant graft 310 could be resected from a native donor tissue structure of any suitable type as a monolithic, single-piece unit comprising the soft tissue 312 and natively attached bone block 320.

Alternatively, and particularly when the transplant graft 310 is at least partially manufactured, rather than simply harvested from a native donor tissue, the bone block 320, soft tissue 312, and/or any components of the transplant graft could be separate during at least a portion of the manufacturing process, but could be combined during that process to become a unitary whole by the time manufacture is complete. Regardless of the manner in which such is provided, it may be desirable, for some embodiments of the present invention, for the bone block 320 and soft tissue 312 to be attached together in at least a semi-permanent manner, such that one of these structures would have to be cut or broken to separate them.

The transplant graft 310 may be at least partially made of any suitable material or combination of materials, including but not limited to bone, soft tissue, plastics, metals, composite materials, shape-memory materials, elastic materials, woven materials, extruded materials, and/or any other suitable manmade or nature-provided material(s).

The transplant graft 310, and components thereof, may have any suitable dimensions. For example, for an MUCL replacement, the soft tissue 312 may be in the range of about 30-50 mm long, more particularly about 40 mm, and about 6-10 mm wide, more particularly about 8 mm. Likewise, the bone block 320 may be in the range of about 10-30 mm long, more particularly about 20 mm, and about 3-9 mm wide, more particularly about 5 mm. Optionally, and as shown in FIG. 3A, the second soft tissue end 312 may be bifurcated, for example, for about the last 15 mm of the total soft tissue 312 length. Optionally, any portion(s) of the transplant graft 310 may include any desirable integrally formed or separately provided reinforcing structures, such as aglets, stay sutures, basting stitches, caps, or any other means for reinforcing any desired structure of the transplant graft.

The bone block 320 or another portion of the transplant graft 310 may include one or more fastener holes 322 having any desired placement, shape, size, or other physical characteristics. These fastener holes 322 may be used to aid in attaching the transplant graft 310 to an anchor cavity of the receiving bone, as will be discussed below.

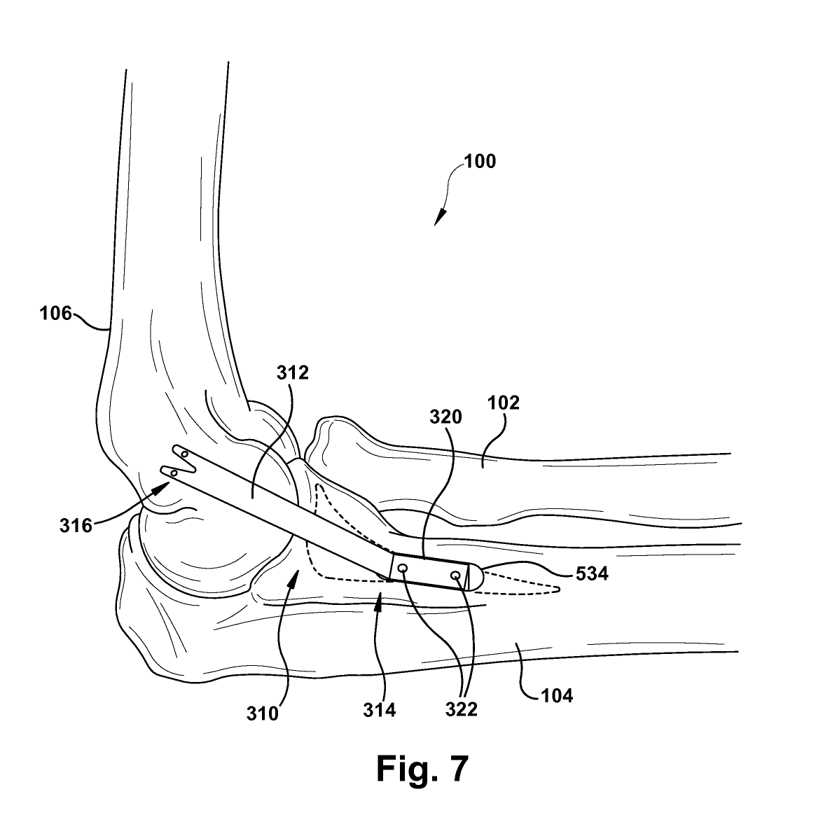

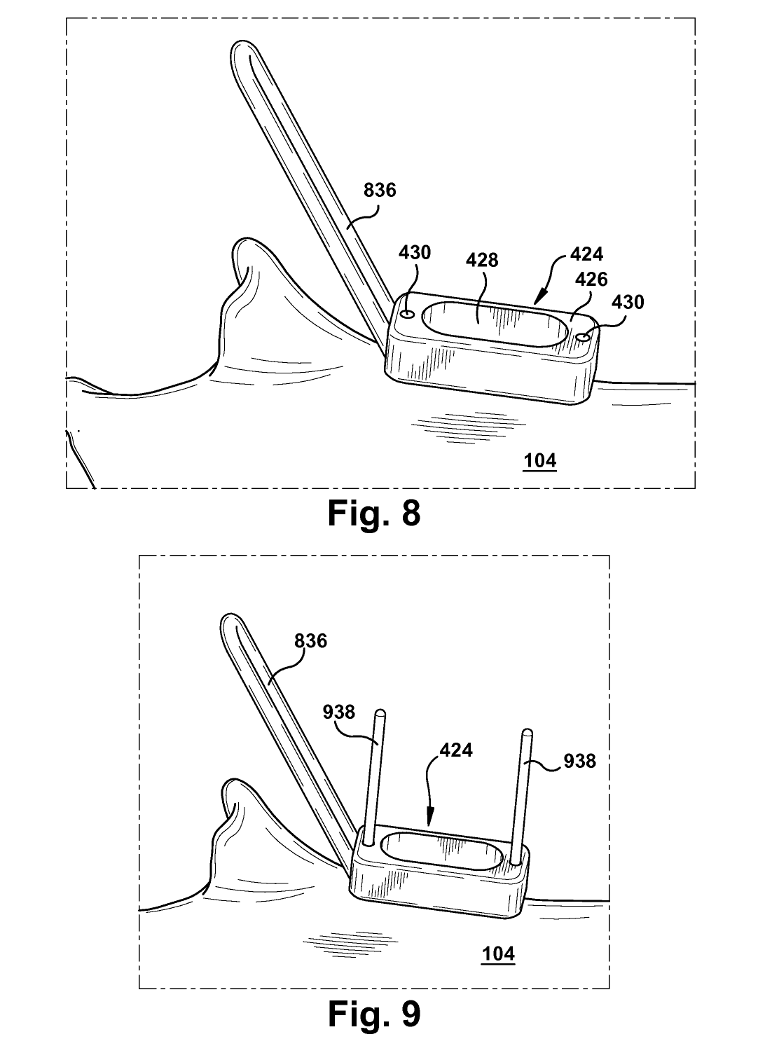

FIGS. 4A-4B depict a cavity template 424 for guiding a material-removal tool to create the anchor cavity in the receiving bone. The cavity template 424 comprises a sidewall 426, open at the top and bottom to define a surrounded volume 428 through which a material-removal tool can be passed to come into guided contact with the underlying receiving bone. At least one anchoring hole 430 may be provided in the cavity template 424 to accept a guide pin or other anchoring structure to hold the cavity template in position with respect to the receiving bone during formation of the anchor cavity. The cavity template 424 may also or instead include one or more template protrusions (not shown), such as spikes or pegs, which can interact with the receiving bone and help prevent unwanted displacement of the cavity template during generation of the anchor cavity. While the cavity template 424 will be substantially rigid in many applications of the present invention to resist deforming under pressure from the material-removal tool, it is also contemplated that the cavity template may include one or more flexible portions to allow the user to create a desired custom anchor cavity shape.

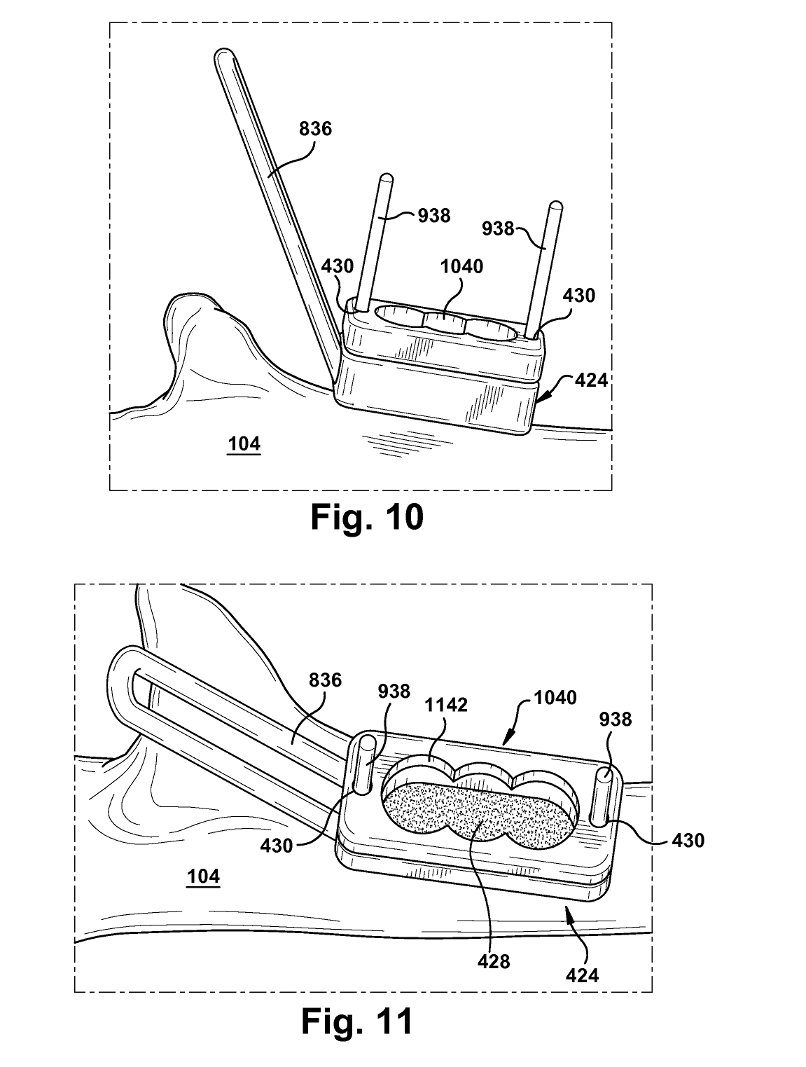

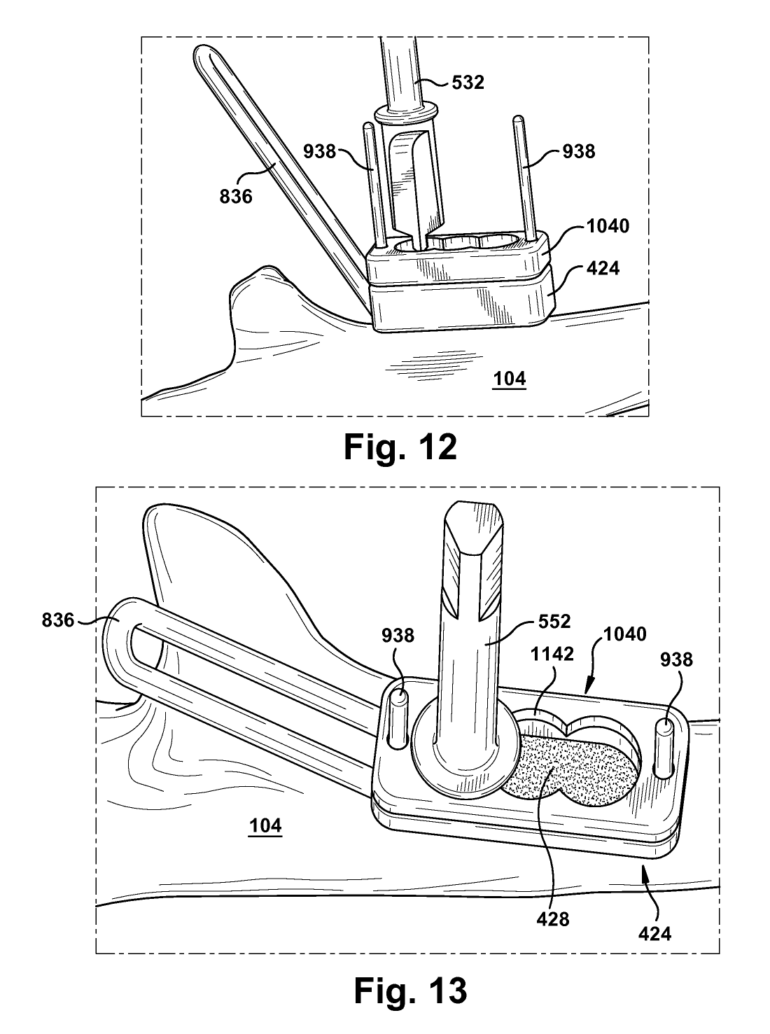

Operation of the cavity template 424 is shown in FIGS. 5A-5B. In these Figures, the cavity template 424 has been placed into a predetermined relationship with a receiving bone, shown here as an ulna 104. The cavity template 424 includes a plurality of anchoring holes 430 to accept corresponding pins or other temporary fasteners therethrough for anchoring the cavity template to the ulna 104. However, those fasteners have been omitted from FIGS. 5A-5B for clarity.

Once the cavity template 424 has been placed and secured as desired with respect to the receiving bone, as shown in FIG. 5A, a material-removal tool 532 of any desired type may be inserted into the surrounded volume 428 (shaded in FIG. 5A), as shown in FIG. 5B. The material-removal tool 532 may then be brought into penetrating, machining contact with the receiving bone, guided by the sidewall 426 or another structure of the cavity template 424, in order to form a desired anchor cavity 534. For example, when a router (not shown) is the material-removal tool 532, the sidewall 426 could be made of stainless steel or another router-resistant material and the user will simply run the router around inside the sidewall, visually and/or physically guided by the cavity template 424. Any suitable manual or powered material-removal tool(s) 532 may be provided, such as, but not limited to, a router, a mill, a drill, a bovie, a scalpel or other blade, a cannulated or solid reamer, a curette, an awl, and/or any other desired tool. Optionally, a height of the cavity template 424 (the vertical dimension, in the orientation of FIG. 5B) could be designed such that the material-removal tool 532 is limited in insertion depth by interaction with some structure of the material-removal tool and the top of the sidewall 426 in a depth-stop type manner.

It is also contemplated that the material-removal tool(s) 532 and/or the surrounded volume 428 could be designed and/or chosen to have corresponding dimensions for efficient machining. For example, the surrounded volume 428 could be shaped/sized such that a single one-way pass, or a single circuit, of the material-removal tool 532 within the cavity template 424 will cause the removal of all of the desired material, thus obviating the need for the user to go back and “clean up” kerf or other undesired extra material in the anchor cavity 534.

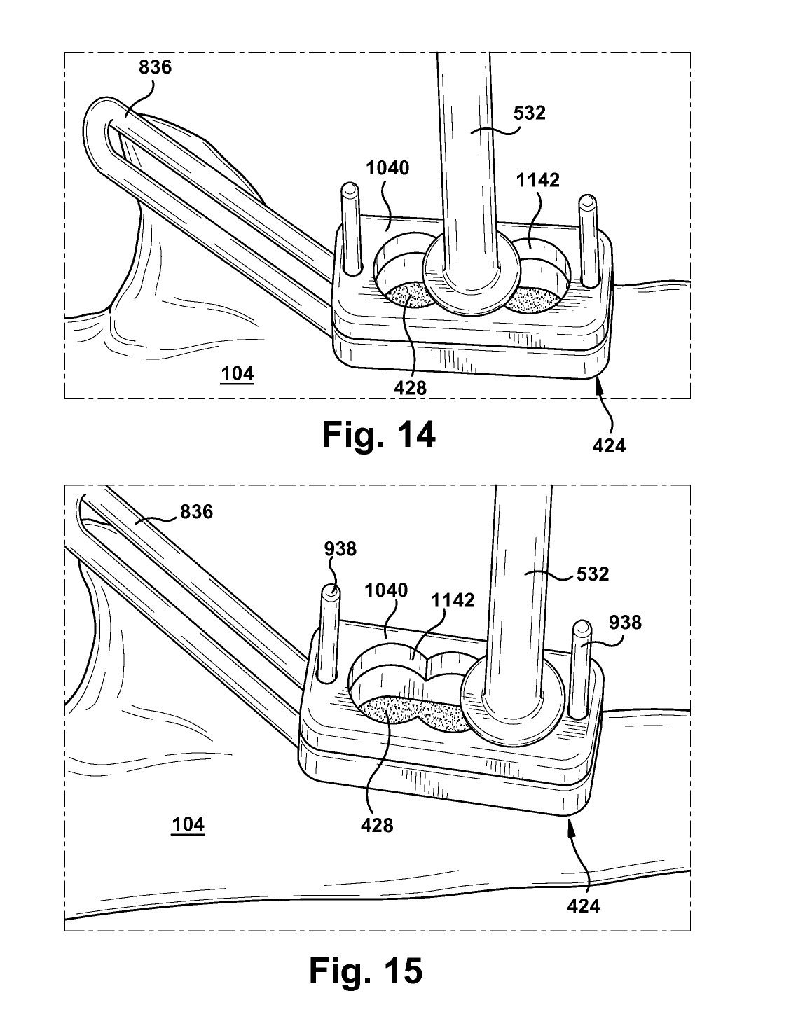

When the anchor cavity 534 has been machined as desired, the cavity template 424 can be removed from the receiving bone and either cleaned/prepared for reuse or disposed of. Removal of the cavity template 424 leaves the receiving bone (ulna 104) and anchor cavity 534 in a condition to facilitate further steps of the inventive system, as shown schematically in FIG. 6. As can be seen in this Figure, the anchor cavity 534 is shaped to substantially accept the bone block (omitted from FIG. 6) in a mating relationship. The term “mating” is used herein to indicate a relationship in which the contours of two structures are at least partially matched or coordinated in at least two dimensions. For example, both the anchor cavity 534 and the bone block 320 could have profiles that are concavely curved, convexly curved, planar/linear, or any combination of those or other profile shapes.

Stated differently, the bone block 320 could have a graft form factor, and the cavity template 424 could embody at least a portion of the graft form factor which, when the cavity template is used with an appropriate material-removal tool 532, correlates at least one dimension of the resulting anchor cavity with the graft form factor. As an example, if the bone block 320 has a graft form factor with a width of 6 mm, the cavity template 424 could have a width of 6.2 mm or otherwise guide the material-removal tool 532 to create an anchor cavity 534 with a width of 6.2 mm. Accordingly, this example anchor cavity 534 would have a width configured to closely accept the bone block 320 therein, with clearance of 0.1 mm on either side of the width of the bone block. For many applications of the present invention, the user will want the anchor cavity 534 to be slightly larger than the bone block 320, for ease of insertion. However, it is contemplated that exactly matching, or even slightly smaller, dimensions of the anchor cavity 534 as compared to the bone block 320 may facilitate a desired friction- or interference-fit of the bone block 320 within the anchor cavity 534 for some applications of the present invention. It is also contemplated that the components making up the system disclosed herein may be designed with correlated relative dimensions chosen for a desired end result (e.g., to allow for dimensional tolerances or slight inaccuracies in measurements), rather than requiring the relative dimensions to be identical among the various components. Optionally, additional cavities, holes, tunnels, channels, protrusions, or other features may be machined into or provided to the receiving bone (ulna 104) or bone block 320 during preparation of these structures.

Once the anchor cavity 534 has been machined and otherwise prepared as desired, at least a portion of the bone block 320 may be placed in the anchor cavity. For example, a majority of the volume of the bone block 320 may be inserted into the anchor cavity 534 such that a relatively small amount of the bone block protrudes above the surrounding receiving bone surface. It is also contemplated that the anchor cavity 534 could have a depth chosen such that the bone block 320 is completely retracted into the anchor cavity, flush with or even below the surrounding receiving bone surface.

Optionally, a portion of the bone block 320 may protrude from the anchor cavity 534 in an asymmetrical or canted manner, such that the end of the bone block having the attached soft tissue 312 is substantially above the surface of the surrounding receiving bone, but the opposite end of the bone block is substantially flush with or even recessed below the surrounding receiving bone surface. When such a “ramp-like” anchor cavity 534 profile is desired, the sidewall 426 of the cavity template 424 may be shaped to echo the desired anchor cavity bottom slant and thus provide a variable depth-stop function during the machining process

The bone block 320, once placed in the anchor cavity 534 as desired, is then fastened within the anchor cavity. This fastening may be accomplished in any desired manner. For example, an adhesive substance (e.g., bone cement or the like) may be used to adhere the bone block 320 within the anchor cavity 534. As another example, at least one penetrating fastener (not shown) may be passed through at least a portion of the bone block 320 and into the receiving bone. This latter example may be particularly applicable when the bone block 320 includes at least one fastener hole 322, though the bone block could also or instead be directly penetrated by a fastener without the benefit of a previously provided fastener hole. Other examples of suitable fasteners include, but are not limited to, bone screws (cannulated and/or solid), with or without washers; suture anchors (screw-in and tap-in types); interference screws; and suspensory fixation devices.

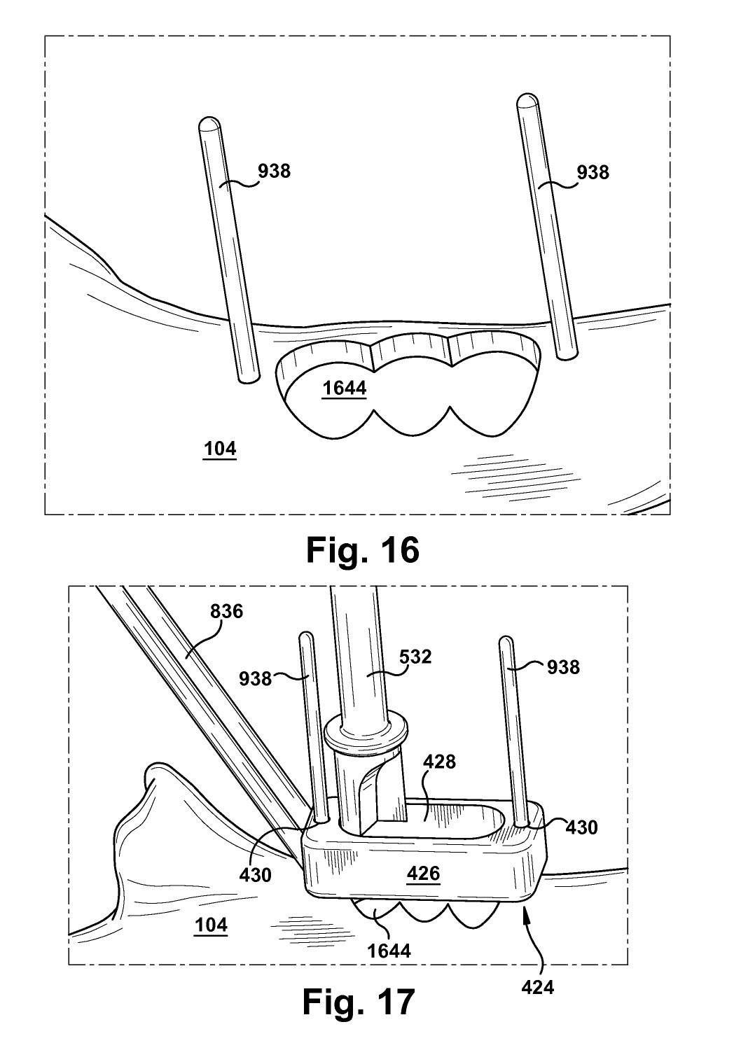

Once the transplant graft 310 has been fastened into place as desired (such as through the fastening of the bone block 320 in the anchor cavity 534), the soft tissue 312—and particularly the first soft tissue end 314—will have achieved a predetermined connected relationship with the receiving bone, such as with the ulna 104 shown in FIG. 7. In most applications of the present invention, the second soft tissue end 316 will still be unattached to any structures at this stage. Accordingly, the user can fasten the second soft tissue end 316 in any suitable manner to another structure within the patient's body such as, but not limited to, the receiving bone, an artificial structure previously provided to the patient (e.g., a prosthesis), and/or a native structure of the patient anatomy. This fastening of the second soft tissue end 316 may be made at a location spaced apart from the anchor cavity 534, such as the attachment to the humerus 106 shown in FIG. 7. FIG. 7, accordingly, depicts a final arrangement of a transplant graft 310 as a replacement MUCL 108.

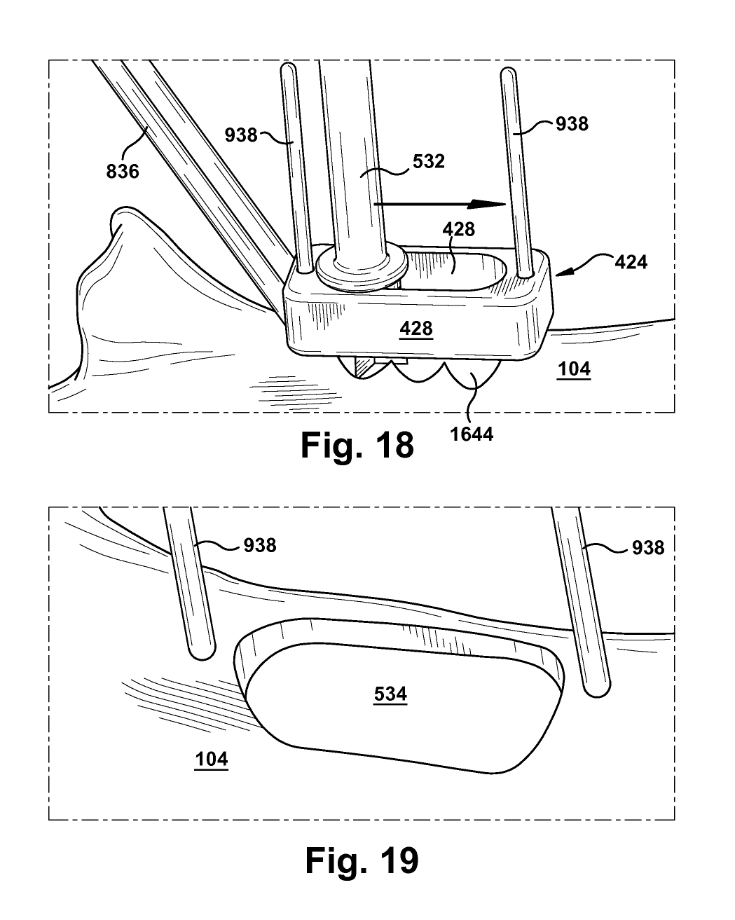

FIGS. 8-19 schematically depict a sequence of operation of an apparatus similar to that previously discussed, but having additional components, as will be set forth below. In FIG. 8, a cavity template 424 having an attached manipulation handle 836 has been placed (optionally, with the assistance of the manipulation handle) into a predetermined relationship with a receiving bone, shown here as an ulna 104. Though depiction of such is omitted in the Figures for clarity, the manipulation handle 836 may be held manually or automatically (e.g., attached to a retractor) during use of the cavity template 424 to maintain the depicted predetermined relationship.

In FIG. 9, temporary fastener pins 938 have been inserted through the anchoring holes 430 of the cavity template 424 to assist with maintaining the cavity template in the predetermined relationship with the ulna 104.

FIG. 10 shows the arrangement of FIG. 9 with the addition of an optional tool guide 1040. Here, the tool guide 1040 includes a pair of anchoring holes 430 which align with those of the cavity template 424 so that the user can slide the tool guide over the previously placed fastener pins 938 and into a desired position with respect to the cavity template 424. As can be seen in FIG. 11, the tool guide 1040 includes a triple-lobed guiding aperture 1142 which provides access through the body of the tool guide and into the underlying surrounded volume 428 (shaded in FIG. 11) of the cavity template 424. A tool guide 1040, when present, can assist the user with operating the material-removal tool 532 to form the anchor cavity 534.

Use of the tool guide 1040 with a hole-forming material-removal tool 532 (e.g., a drill or circular reamer) is shown in the sequence of FIGS. 12-15. In these Figures, the tool guide 1040 and cavity template 424 have already been placed as desired. The material-removal tool 532 is then guided, by insertion through the guiding aperture 1142, to extend through the surrounded volume 428 of the cavity template 424 and thereby remove bone from the ulna 104 in three overlapping hole locations. It is contemplated that the widest dimension of each section of the guiding aperture 1142 will be coordinated with the outer dimension of the material-removal tool 532 such that the material-removal tool does not “skip” laterally (e.g., left-to-right in the orientation of FIGS. 13-15) from one lobe of the guiding aperture to another. It is contemplated that the tool guide 1040 will be at least partially comprised of a material that is resistant to damage caused by undesirable contact between the material-removal tool 532 and the tool guide 1040. The tool guide 1040, as with all components of the present invention, may be reusable or disposable, patient-specific or generic.

Because of the spacing of the three lobes of the guiding aperture 1142, the series of three holes correspondingly machined by the material-removal tool 532 will extend contiguously in an overlapping relationship, as shown in FIG. 16. In FIG. 16, the tool guide 1040 and cavity template 424 have been removed to show the manner in which the three holes drilled in FIGS. 13-15 combine to form an intermediate cavity 1644 in the receiving bone (ulna 104). However, the tool guide 1040 and/or cavity template 424 can be left in place, with no exposure of the intermediate cavity 1644. When the tool guide 1040 and/or cavity template 424 is removed, as shown in FIG. 16, the temporary fastener pins 938 may be left in situ to later help guide structures into a predetermined relationship with the receiving bone.

Optionally, the bone block 320 could be machined for mating acceptance by the intermediate cavity 1644—if so, the intermediate cavity can be considered to serve as an anchor cavity 534. However, for many applications of the present invention, further modifications will be made to the intermediate cavity 1644 before the bone block 320 is accepted. Turning to FIG. 17, the cavity template 424 is in the predetermined relationship with the receiving bone, either having been replaced there or never having been removed as in the FIG. 16 view. Regardless of how the predetermined relationship is achieved, the cavity template 424 is configured to accept a material-removal tool 532, which may be the same as that shown in FIGS. 12-15 or may be different. The material-removal tool 532 is then positioned and re-positioned within the surrounded volume 428 as desired (similar to the arrangement shown in FIG. 5B), as shown in FIG. 18. For example, the material-removal tool 532 of FIGS. 17-18 may have an outer diameter/dimension slightly smaller than a width of the surrounded volume 428 such that the sidewall 428 of the cavity template 424 guides removal of material within the footprint of the surrounded volume 428 with a single pass of the material-removal tool.

As can be seen in the Figures, manipulation of the material-removal tool 532 of FIGS. 17-18, guided by the cavity template 424, alters the intermediate cavity 1644 to create the anchor cavity 534. That is, as shown in FIG. 19, the material-removal tool 532 is guided by the cavity template 424 to smooth out the scalloped outline of the intermediate cavity 1644 to produce the final anchor cavity 534. For example, suitable dimensions for an ovoid anchor cavity 534, as shown in a particular use environment could be approximately eight millimeters wide, twenty millimeters long, and five millimeters deep. Regardless of dimensions, though, once the anchor cavity 534 is formed, the cavity template 424 (along with, optionally, any associated fastener pins 938) can be removed from the receiving bone (ulna 104 here) and the surgical procedure then will proceed apace, such as through installation of a bone block 320 as previously described.

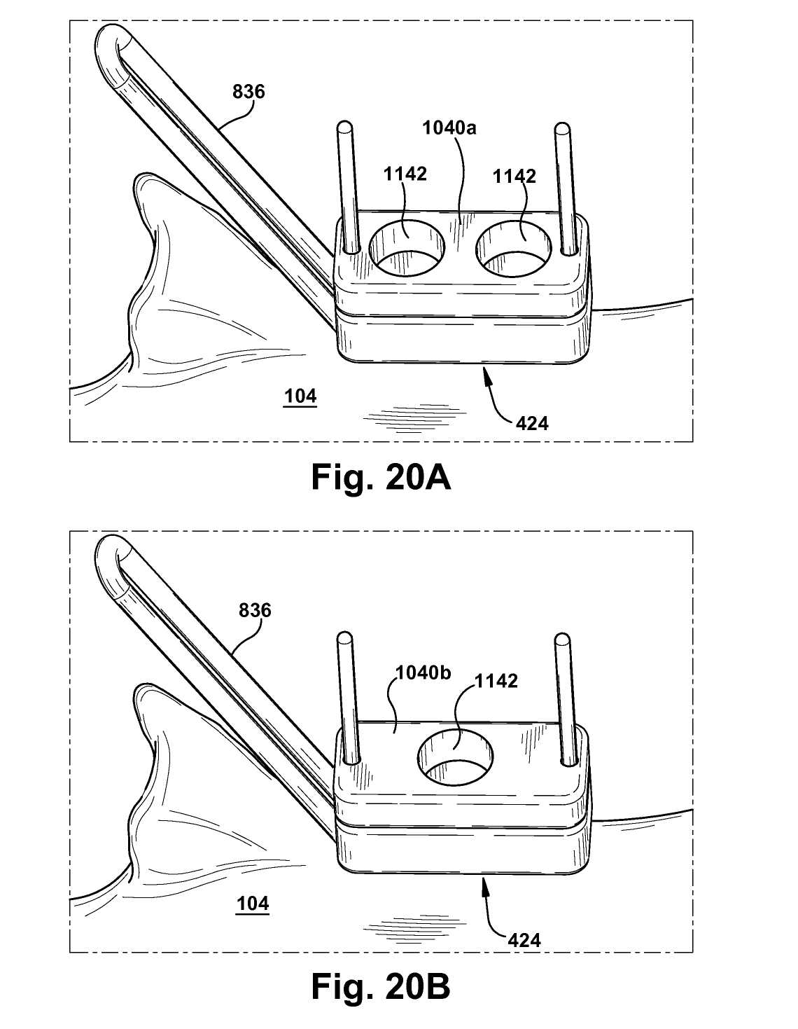

FIGS. 20A and 20B depict optional alternate embodiments of tool guides 1040a and 1040b which can be used, singly or together, to guide a material-removal tool 532 in creating the intermediate cavity 1644, particularly if, for some reason, the triple-lobed configuration of the aforementioned tool guide 1040 is not used. For example, the tool guides 1040a and 1040b can be used in sequence, along with an appropriate material-removal tool 532, to produce the triple-lobed intermediate cavity 1644 shown in FIG. 16. This may be particularly useful if the “partial circumference” guiding of each of the three holes of the tool guide 1040 of FIG. 10 is not sufficient to constrain the material-removal tool 532 and full-circumference guiding is desired for each of the three holes making up the triple-lobed intermediate cavity 1644.

While aspects of the present invention have been particularly shown and described with reference to the preferred embodiment above, it will be understood by those of ordinary skill in the art that various additional embodiments may be contemplated without departing from the spirit and scope of the present invention. For example, the specific methods described above for using the transplant graft 310 or other portions of the system are merely illustrative; one of ordinary skill in the art could readily determine any number of tools, sequences of steps, or other means/options for placing the above-described apparatus, or components thereof, into positions substantively similar to those shown and described herein. Radiopaque markers could be placed in association with any of the described structures and components as desired. Any of the described structures and components could be integrally formed as a single unitary or monolithic piece or made up of separate sub-components, with either of these formations involving any suitable stock or bespoke components and/or any suitable material or combinations of materials; however, the chosen material(s) should be biocompatible for many applications of the present invention. The mating relationships formed between the described structures need not keep the entirety of each of the “mating” surfaces in direct contact with each other but could include spacers or holdaways for partial direct contact, a liner or other intermediate member for indirect contact, or could even be approximated with intervening space remaining therebetween and no contact. Though certain components described herein are shown as having specific geometric shapes, all structures of the present invention may have any suitable shapes, sizes, configurations, relative relationships, cross-sectional areas, or any other physical characteristics as desirable for a particular application of the present invention. The transplant graft 310 or other portions of the system may include a plurality of structures cooperatively forming any components thereof and temporarily or permanently attached together in such a manner as to permit relative motion (e.g., pivoting, sliding, or any other motion) therebetween as desired. While discrete fasteners are discussed herein, it is contemplated that fasteners connected to one another in any desired manner may also or instead be used with the present invention. A cavity template 424 is shown here as accepting a material-removal tool 532 into a central aperture thereof, but it is also contemplated that the cavity template could also or instead include “stencil”-type feature(s) around an outside of which the material-removal tool could be passed. During formation of the anchor cavity 534, any desired “finishing” work (e.g., cleaning up or smoothing the inner surfaces of the anchor cavity) may be performed, whether or not the cavity template 424 remains in place during those finishing tasks. A tool guide 1040 could be used with a cavity template 424 in parallel, as shown in the Figures, or the tool guide could instead be used on its own, optionally in series with a cavity template--in the latter arrangement, the tool guide could itself be considered a cavity template. Any structures or features described with reference to one embodiment or configuration of the present invention could be provided, singly or in combination with other structures or features, to any other embodiment or configuration, as it would be impractical to describe each of the embodiments and configurations discussed herein as having all of the options discussed with respect to all of the other embodiments and configurations. A device or method incorporating any of these features should be understood to fall under the scope of the present invention as determined based upon the claims below and any equivalents thereof.

Other aspects, objects, and advantages of the present invention can be obtained from a study of the drawings, the disclosure, and the appended claims.

Having described the invention, we claim:

1. A method of providing a soft-tissue transplant to a receiving bone, the method comprising the steps of:
providing a transplant graft comprising an elongated soft tissue, having first and second soft tissue ends longitudinally separated by a soft tissue body, and a bone block directly connected with the first soft tissue end, the bone block and soft tissue having been integrally formed as a unitary whole;
machining an anchor cavity in the receiving bone, the anchor cavity being shaped to substantially accept the bone block in a mating relationship;
placing a majority of a volume of the bone block in the anchor cavity; and
fastening the bone block within the anchor cavity.

2. The method of claim 1, including the step of resecting the transplant graft from a native donor tissue.

3. The method of claim 1, including the step of creating the transplant graft as a unitary whole at least partially formed of a manmade material.

4. The method of claim 1, wherein the step of fastening the bone block within the anchor cavity includes the step of adhering the bone block within the anchor cavity with an adhesive substance.

5. The method of claim 1, wherein the step of fastening the bone block within the anchor cavity includes the step of passing at least one penetrating fastener through at least a portion of the bone block and into the receiving bone.

6. The method of claim 1, wherein the bone block and soft tissue are integrally formed as a unitary whole as native tissue structures of a donor patient, with the first soft tissue end being anchored to the bone block as a feature of the native tissue anatomy.

7. The method of claim 1, wherein the step of machining an anchor cavity in the receiving bone includes the steps of:
placing a cavity template into a predetermined relationship with the receiving bone; and
guiding a material-removal tool, with the cavity template, to create the anchor cavity.

8. The method of claim 7, wherein the bone block has a graft form factor and the cavity template embodies at least a portion of the graft form factor to correlate at least one dimension of the anchor cavity with the graft form factor.

9. The method of claim 1, wherein the step of machining an anchor cavity in the receiving bone includes the steps of:
placing a tool guide into a predetermined relationship with the receiving bone;
guiding a material-removal tool, with the tool guide, to create an intermediate cavity;
placing a cavity template into a predetermined relationship with the receiving bone; and
guiding a material-removal tool, with the cavity template, to create the anchor cavity via alteration of the intermediate cavity.

10. The method of claim 1, including the step of fastening the second soft tissue end to a patient anatomy native structure at a location spaced apart from the anchor cavity.

11. A system for providing a soft-tissue transplant to a receiving bone, the system comprising:
a transplant graft comprising an elongated soft tissue, having first and second soft tissue ends longitudinally separated by a soft tissue body, and a bone block directly connected with the first soft tissue end, the bone block and soft tissue having been integrally formed as a unitary whole; and
an anchor cavity in the receiving bone, the anchor cavity being shaped to substantially accept the bone block in a mating relationship;
wherein a majority of a volume of the bone block is placed in the anchor cavity and the bone block is fastened to the receiving bone to place the soft tissue in a predetermined connected relationship with the receiving bone.

12. The system of claim 11, wherein the transplant graft is resected from a native donor tissue.

13. The system of claim 11, wherein the transplant graft is created as a unitary whole at least partially formed of a manmade material.

14. The system of claim 11, wherein an adhesive substance is used to fasten the bone block to the receiving bone.

15. The system of claim 11, wherein at least one penetrating fastener is passed through at least a portion of the bone block and into the receiving bone to fasten the bone block to the receiving bone.

16. The system of claim 11, wherein the bone block and soft tissue are integrally formed as a unitary whole as native tissue structures of a donor patient, with the first soft tissue end being anchored to the bone block as a feature of the native tissue anatomy.

17. The system of claim 11, including a cavity template for guiding a material-removal tool to create the anchor cavity with a cavity form factor configured to accept at least a portion of the bone block.

18. The system of claim 17, including a tool guide for guiding a material-removal tool to create an intermediate cavity, the intermediate cavity being altered by a material-removal tool guided by the cavity template to create the anchor cavity.

19. The system of claim 17, wherein the bone block has a graft form factor and the cavity template embodies at least a portion of the graft form factor to correlate at least one dimension of the cavity form factor with the graft form factor.

20. The system of claim 11, wherein the transplant is an autologous transplant.

21. A method of providing a medial ulnar collateral ligament transplant graft to an ulna, the method comprising the steps of:
providing a transplant graft comprising an elongated ligament, having first and second ligament ends longitudinally separated by a ligament body, and a bone block directly connected with the first ligament end, the bone block and ligament having been integrally formed as a unitary whole;
machining an anchor cavity in the ulna, the anchor cavity being shaped to substantially accept the bone block in a mating relationship;
placing a majority of a volume of the bone block in the anchor cavity; and
fastening the bone block within the anchor cavity.

22. The method of claim 21, including the step of resecting the transplant graft from a native donor tissue.

23. The method of claim 21, including the step of creating the transplant graft as a unitary whole at least partially formed of a manmade material.

24. The method of claim 21, wherein the bone block and soft tissue are integrally formed as a unitary whole as native tissue structures of a donor patient, with the first ligament end being anchored to the bone block as a feature of the native tissue anatomy.

25. The method of claim 21, wherein the step of machining an anchor cavity in the ulna includes the steps of:
placing a cavity template into a predetermined relationship with the ulna; and
guiding a material-removal tool, with the cavity template, to create the anchor cavity.

26. The method of claim 21, wherein the step of machining an anchor cavity in the ulna includes the steps of:
placing a tool guide into a predetermined relationship with the ulna;
guiding a material-removal tool, with the tool guide, to create an intermediate cavity;
placing a cavity template into a predetermined relationship with the ulna; and
guiding a material-removal tool, with the cavity template, to create the anchor cavity via alteration of the intermediate cavity.

27. The method of claim 25, wherein the bone block has a graft form factor and the cavity template embodies at least a portion of the graft form factor to correlate at least one dimension of the anchor cavity with the graft form factor.

28. The method of claim 21, including the step of fastening the second ligament end to a humerus operatively connected to the ulna.

29. The method of claim 21, wherein the transplant graft is acquired autologously.
